(19) United States
(12) Patent Application Publication
(10) Pub. No.: US 2025/0360119 A1
(43) Pub. Date: Nov. 27, 2025

(54) THIAZOLIDINEDIONES FOR THE TREATMENT OF MUSCULAR DYSTROPHIES

(71) Applicants: The Johns Hopkins University, Baltimore, MD (US); University of Florida Research Foundation, Incorporated, Gainesville, FL (US)

(72) Inventors: Laszlo Nagy, Baltimore, MD (US); Lee Sweeney, Gainesville, FL (US); David Hammers, Gainesville, FL (US); Andreas Patsalos, Baltimore, MD (US)

(21) Appl. No.: 18/874,975

(22) PCT Filed: Jun. 15, 2023
(86) PCT No.: PCT/US2023/025437

§ 371 Date: Dec. 13, 2024

Related U.S. Application Data
(60) Provisional application No. 63/352,464, filed on Jun. 15, 2022.

(51) Int. Cl. A61K 31/4439; A61P 21/00

(52) U.S. Cl. CPC A61K 31/4439; A61P 21/00

(57) ABSTRACT

Methods for treating a disease, condition, or disorder associated with impaired muscle regeneration, including a muscular dystrophy, such as Duchenne muscular dystrophy (DMD) and Becker muscular dystrophy (BMD), or limb-girdle muscular dystrophy (LGMD), by administering a PPARγ agonist to a subject. The PPARγ agonist can be one or more thiazolidinediones including, but not limited to pioglitazone and rosiglitazone.

### BACKGROUND

Duchenne muscular dystrophy (DMD) is a lethal, childhood-onset degenerative muscle disease caused by genetic mutations leading to the loss of dystrophin, a protein that stabilizes the integrity of muscle during contractile activity. Becker muscular dystrophy (BMD) is a milder form of this disease caused by dystrophin mutations that result in a truncated protein. Both diseases result in the progressive loss of musculature that is replaced by fibrosis, leading to mobility impairments and loss of ambulation. Although there are ongoing drug and gene therapy efforts that aim to convert the lethal DMD to a milder BMD-like disease, identifying effective therapeutics that can slow, prevent, or even reverse the replacement of muscle in DMD, BMD and a number of other diseases remains a significant unmet clinical need for patients afflicted by these diseases.

### SUMMARY

In some aspects, the presently disclosed subject matter provides a method for treating a disease, condition, or disorder associated with impaired muscle regeneration, the method comprising administering a PPARγ agonist to a subject in need of treatment thereof.

In certain aspects, the PPARγ agonist comprises one or more thiazolidinediones. In particular aspects, the one or more thiazolidinediones is selected from pioglitazone and rosiglitazone.

In certain aspects, the disease, condition, or disorder associated with impaired muscle regeneration is selected from a muscular dystrophy, an inflammatory muscle disease, trauma or injury, and aging.

In particular aspects, the muscular dystrophy is selected from Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD), myotonic mystrophy (MMD), oculopharyngeal muscular dystrophy (OPMD), distal muscular dystrophy (DD), and congenital muscular dystrophy (CMD).

In particular aspects, the inflammatory muscle disease is selected from polymyositis, dermatomyositis, inclusion body myositis, juvenile myositis, and necrotizing autoimmune myopathy.

In certain aspects, the disease, condition, or disorder is at an early stage of disease progression. In certain aspects, the disease, condition, or disorder is at a late stage of disease progression.

In certain aspects, administrating the PPARγ agonist improves one or more of muscle function, muscle structure, muscle fiber cross-sectional area, muscle regeneration, and cardiopulmonary function of the subject.

In certain aspects, administering the PPARγ agonist ameliorates or attenuates one or more of disease progression, fibrosis, necrosis of muscle fiber, and inflammation of the subject.

In certain aspects, administering the PPARγ agonist enhances regenerative macrophage activity in the subject.

In certain aspects, administering the PPARγ agonist promotes a macrophage phenotype transition from pro-inflammatory to pro-regenerative.

In certain aspects, the subject has an age of less than about 5 years, between about 5 years to about 12 years old, between about 12 years to about 15 years, and greater than 15 years.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1 shows a cross-sectional pathobiological analysis of aging D2.mdx cohorts reveal distinct stages of dystrophic progression;

FIG. 2A and FIG. 2B demonstrate that the PPARγ-GDF3 axis is required for muscle regeneration after sterile injury. (FIG. 2A) Schematic representation of the IL-4-STAT6-PPARγGDF3 regulatory axis controlling myoblast fusion. (FIG. 2B) Heatmap representation of lipid contents isolated from injured and regenerating muscles. Adapted from Varga et al., 2016;

FIG. 3A, FIG. 3B, and FIG. 3C show a proof-of-concept trial identifying PPARγ as a valid target in DMD. 4-week treatment protocol of 1- and 2-mo D2.mdx mice with vehicle (n=3) or PPARγ agonist Pioglitazone (Pio; 10 mg/kg; n=3). (FIG. 3A) H&E stain reveals reduced areas of necrotic fibers and inflammation, and (FIG. 3B) increased CSA were observed in Pio-treated gastrocnemius and quadriceps muscles. (FIG. 3C) Pio-treatment improves in vivo muscle function (n=3). *P≤0.05, **P≤0.01, ***P≤0.001;

FIG. 4A, FIG. 4B, FIG. 4C demonstrate that pioglitazone treatment decreases fibrosis and increases regeneration in aged D2.mdx diaphragm. D2.mdx mice began daily vehicle or pioglitazone (10 mg/kg) treatments 8 months of age (n=4), and were evaluated for muscle fibrosis and muscle regeneration after 4 weeks of treatment. (FIG. 4A) Representative images of Masson's trichrome staining (fibrosis stained in blue) and immunofluorescent staining for embryonic myosin (eMyHC; regenerating fibers) and CD68 (macrophages) in vehicle and pioglitazone-treated diaphragm sections. Scale bars indicate 100 μm. Quantification of (FIG. 4B) fibrosis and (FIG. 4C) regenerating muscle fibers in vehicle and pioglitazone-treated diaphragm sections. Values indicate mean ±SEM. Data were analyzed using T-tests (α=0.05; effect size reported as Cohen's d; ****p<0.0001 vs. Vehicle);

FIG. 5 demonstrates that PPARγ activation is linked to the functional improvement of established and emerging DMD therapeutics. Upstream regulator analysis (IPA) of common differentially regulated genes in D2.mdx mice treated with prednisolone or givinostat predicts PPARγ and its agonists to control genes related to the positive effect of these drugs on muscle function;

FIG. 6 shows muscle disease in mouse models of DMD and BMD. Muscles from 4-5 mo wild-type DBA/2J, D2.mdx (mdx), and D2.mdx expressing a skeletal muscle specific micro-dystrophin transgene (mdx-μDysTg) were stained for dystrophin using immunofluorescence (IF) and fibrosis using picrosirius red staining. Both dystrophic lines exhibit muscle fibrosis, indicative of muscle pathology. Muscle macrophages, identified using CD68, of dystrophic muscles exhibit nuclear PPARγ staining, which is not observed in the few resident macrophages observed in wild-type muscle. Scale bars indicate 10 μm;

FIG. 7A and FIG. 7B show immune cell invasion and single-cell expression profiles in D2.mdx mice. (FIG. 7A) Left panel indicates the total number of CD45⁺ invading cells isolated from tibialis anterior and gastrocnemius muscles from control and D2.mdx (2-mo mice); right panel indicates the absolute number of inflammatory (Ly6C^{high} F4/80^{low}) and repair (Ly6C^{low} F4/80^{high}) MFs present in the dystrophic muscle. P<0.05 (FIG. 7B) Expression of key dystrophic muscle and immune markers as well as critical lipid mediator enzymes in the isolated CD45⁺ cell populations from 2-mo D2.mdx muscles, determined by single cell RNA-seq experiments (5001 single-cell profiles are shown);

FIG. 8A, FIG, 8B, FIG. 8C are spatial transcriptomics experiments, which reveal that Pioglitazone-treatment engages its target and induces known PPARγ target genes and promotes regenerative inflammation of dystrophic muscle. (FIG. 8A) Visualization of the spatial proportion estimates of the identified clusters (k-means) overlaid on the H&E-image of gastrocnemius muscles from D2.mdx (2-mo) vehicle or Pioglitazone-treated mice (10× Visium array, 55-μm spots). Estimated proportions for the 3 clusters, annotated by a pathologist, also is shown. (FIG. 8B) Spatial feature plot of the expression of Myl3 and Spp1, reflecting an increase in regenerative areas and a decrease in inflammatory areas in the Pio-treated samples compared to vehicle, respectively. (FIG. 8C) Dot plot of the expression and abundance of PPARγ target genes reveal target engagement in the Pio-treated samples. Representative inflammatory (decreased with treatment), regenerative (increased with treatment) and GR target genes (unchanged with treatment) also are shown; and

FIG. 9 is a schematic overview showing a focus on early and late activation of macrophage PPARγ in models of DMD and BMD using low (LD) and high (HD) doses of synthetic agonists (pioglitazone or rosiglitazone). Without being bound to any one particular theory, is it thought that short-term PPARγ activation regulates the MF phenotype transition, promotes pro-regenerative gene expression profiles, and reduces fibrosis, while long-term activation will improve muscle and cardiopulmonary function and thus result in amelioration of disease progression.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Thiazolidinediones for the Treatment of Muscular Dystrophies

In some embodiments, the presently disclosed subject matter provides a method for treating a disease, condition, or disorder associated with impaired muscle regeneration, the method comprising administering a PPARγ agonist to a subject in need of treatment thereof. As used herein, a PPARγ agonist activates a peroxisome proliferator-activated receptor-gamma (PPARγ).

In certain embodiments, the PPARγ agonist comprises one or more thiazolidinediones. Thiazolidinediones (TZDs), are a class of heterocyclic compounds consisting of a five-membered C₃NS ring and having the following functional group:

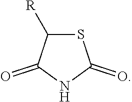

Representative thiazolidinediones include, but are not limited to pioglitazone (ACTOS®), rosiglitazone (AVANDIA®), lobeglitazone, ciglitazone, darglitazone, englitazone, netoglitazone, rivoglitazone, troglitazone, balaglitazone (DRF-2593), AS-605240 [648450-29-7]. The clinical use of many thiazolidinediones, however, have been discontinued. In particular embodiments the one or more thiazolidinediones is selected from pioglitazone and rosiglitazone.

In certain embodiments, the disease, condition, or disorder associated with impaired muscle regeneration is selected from a muscular dystrophy, an inflammatory muscle disease, trauma or injury, and aging.

As used herein, the term “muscular dystrophy” refers to a group of degenerative muscle diseases characterized by gradual weakening and deterioration of skeletal muscles and, in some cases, the heart and respiratory muscles. In particular embodiments, the muscular dystrophy is selected from Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD) (also known as Landouzy-Dejerine), myotonic mystrophy (MMD) (also known as Steinert's Disease), oculopharyngeal muscular dystrophy (OPMD), distal muscular dystrophy (DD), and congenital muscular dystrophy (CMD).

The inflammatory muscle diseases, including myopathies, are a group of diseases, with no known cause, that involve chronic muscle inflammation accompanied by muscle weakness. The majority of these disorders are considered to be autoimmune disorders, in which the body's immune response system that normally defends against infection and disease attacks its own muscle fibers, blood vessels, connective tissue, organs, or joints. These rare disorders may affect both adults and children.

In particular embodiments, the inflammatory muscle disease is selected from polymyositis, which affects skeletal muscles (involved with making movement); dermatomyositis, which includes a skin rash and progressive muscle weakness; inclusion body myositis, which is characterized by progressive muscle weakness and shrinkage; juvenile myositis, and necrotizing autoimmune myopathy, with weakness in the upper and lower body, difficulty rising from low chairs or climbing stairs, fatigue, and muscle pain.

General symptoms of chronic inflammatory muscle diseases include progressive muscle weakness that starts in the proximal muscles, i.e., those muscles closest to the trunk of the body. Other symptoms include fatigue after walking or standing, tripping or falling, and difficulty swallowing or breathing. Polymyositis and dermatomyositis are more common in women than in men. Inclusion body myositis is most common after age 50. Dermatomyositis is more common in children.

In certain embodiments, the disease, condition, or disorder is at an early stage of disease progression. In certain embodiments, the disease, condition, or disorder is at a late stage of disease progression.

In certain embodiments, administrating the PPARγ agonist improves one or more of muscle function, muscle structure, muscle fiber cross-sectional area, muscle regeneration, and cardiopulmonary function of the subject.

In certain embodiments, administering the PPARγ agonist ameliorates or attenuates one or more of disease progression, fibrosis, necrosis of muscle fiber, and inflammation of the subject.

In certain embodiments, administering the PPARγ agonist enhances regenerative macrophage activity in the subject.

In certain embodiments, administering the PPARγ agonist promotes a macrophage phenotype transition from pro-inflammatory to pro-regenerative.

In particular embodiments, the subject has an age of less than about 5 years, including about 0.5, 1, 2, 3, 4, and 5 years, between about 5 years to about 12 years old, including about 5, 6, 7, 8, 9, 10, 11, and 12 years, between about 12 years to about 15 years, including about 12, 13, 14, and 15 years, and greater than 15 years.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a presently disclosed thiazolidinedione and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the presently disclosed thiazolidinediones can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a presently disclosed thiazolidinedione and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + B / {Q}_{B} = Synergy Index ( SI )$$

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### B. Pharmaceutical Compositions and Administration

In another aspect, the present disclosure provides a pharmaceutical composition including one presently disclosed thiazolidinedione alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galacturonic acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed-or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions, and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the presently disclosed thiazolidinedione is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick. As used herein, the term administrating via an “intranasal route” refers to administering by way of the nasal structures.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day.

The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Repurposing Thiazolidinedione for the Treatment of Muscular Dystrophies

### 1.1 Background

Many neuro-muscular diseases involve injury and subsequent tissue remodeling and repair that fails due to inappropriate responses of resident and infiltrating immune cells, leading to disease progression and tissue replacement. Reprogramming the immune cells to allow better repair could potentially slow disease progression and possibly reverse some of the maladaptive remodeling.

Duchenne muscular dystrophy (DMD) is a lethal, childhood-onset degenerative muscle disease caused by genetic mutations leading to the loss of dystrophin, a protein that stabilizes the integrity of muscle during contractile activity. Becker muscular dystrophy (BMD) is a milder form of this disease caused by dystrophin mutations that result in a truncated protein. Both diseases result in the progressive loss of musculature that is replaced by fibrosis, leading to mobility impairments and loss of ambulation. The damage response incurred by dystrophic muscle is not synchronized; therefore, affected musculature chronically contains a mix of both pro-inflammatory and pro-regenerative signals, which ultimately causes discoordination in regenerative inflammation and leads to failed muscle regeneration.

Macrophages (MFs) are cells of the innate immune system that have critical and multifaceted roles in skeletal muscle recovery from injury, including clearance of damaged cellular material and the orchestration of regenerative processes to replace the damaged musculature. The coordination of these events within the affected muscle involves a phenotypic transition of the infiltrating MF population resulting in a shift from a pro-inflammatory profile towards an anti-inflammatory and pro-regenerative phenotype.

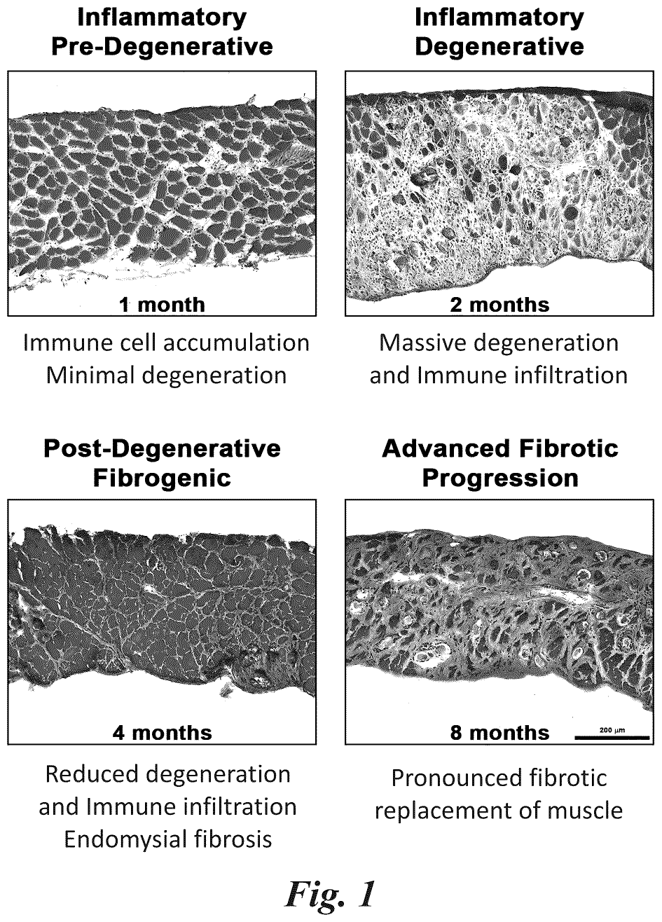

The failed regeneration and fibrotic replacement of functional muscle tissue are prominent features of heritable muscle diseases known as muscular dystrophies (MDs) (FIG. 1). These elements of MD disease can lead to muscle wasting, physical impairments, loss of ambulation, respiratory failure, and, ultimately, death in MD patients. The most common form of MD, Duchenne muscular dystrophy (DMD), is a childhood-onset, X-linked disease caused by mutations in the DMD gene that result in the complete loss of dystrophin, Hoffman et al., 1987, a protein that stabilizes the muscle membrane during contractile activity. Petrof et al., 1993. DMD patients are typically diagnosed by the age of 4, lose ambulation by the age of 12, and succumb to respiratory or heart failure by the age of 30.

Becker MD (BMD) is a milder disease caused by DMD mutations resulting in truncated dystrophin molecules, England et al., 1990; however, it is still a debilitating disease that progresses to the fibrotic replacement of muscle and heart failure. Barp et al., 2017; Cripe and Tobias, 2013. Currently, there are limited treatment options for patients suffering from these diseases, and effective small molecule therapies capable of slowing or reversing muscle loss and fibrotic progression are a major clinical need.

A new era in the management of DMD has recently begun with the initiation of AAV gene therapy trials for DMD. Duan, 2018. These therapies are anticipated to be transformative but with a range of impacts dependent on the patient treatment age and the ability to restore the missing function to all cell types affected. This strategy is complicated by the fact that it aims to replace full-length dystrophin with truncated dystrophin (either a micro-dystrophin construct or a product of exon skipping). Micro-dystrophin therapy has the advantage of being applicable to almost all DMD patients, but with the potential disadvantage of not being as corrective as some products of exon skipping. Thus, the goal from the outset is to create a milder form of DMD that lies on the spectrum of BMD. Therefore, successful implementation of gene therapy for the treatment of DMD also will require efficacious adjunct therapies to control the continual inflammation, progression of fibrosis, and muscle weakness that is exhibited by BMD patients.

DMD is caused by loss of dystrophin in the membrane cytoskeleton of muscle fibers. Hoffman et al., 1987. Affected myofibers show increased plasma membrane instability and can undergo cell death. Necrotic fibers can regenerate through satellite cell activation, myoblast proliferation, fusion, and maturation in about two weeks. Muscle regeneration, however, gradually fails in DMD patients leading to extensive fibrosis and fatty replacement of muscle. Many of the secondary pathological features of DMD also are due to the complex interactions with the surrounding tissue environment. Kharraz et al., 2014.

A key component of this microenvironment is the immune cell infiltration, secreted cytokines and lipid mediators, and the resulting immune phenotypic switches. De Paepe et al., 2012; De Paepe et al., 2021. The immune infiltrate contains a large amount of innate immune cells, including monocytes, macrophages, neutrophils, and dendritic cells. Monocyte-derived macrophages (MFs) are positioned at the crossroads leading to acute inflammation, tissue repair, or regeneration. They coordinate and link the acute inflammatory response, the clearance of necrotic cells during resolution of inflammation, to the promotion of tissue growth. Thus, these cells assume a spectrum of phenotypes and carry out first inflammatory functions and later tissue reparative roles. Recently we attempted to resolve how global profiles in regulatory factors and surface markers define these MF subtypes during a process we designated and termed as regenerative inflammation, resulting in complete restoration of skeletal muscle. Patsalos et al., 2021; Patsalos et al., 2022.

In addition, the phenotype transition and its timing are critical in determining the functional and morphological outcome of regeneration, and it is regulated by endogenous microenvironmental cues. Importantly, the prolonged presence of pro-inflammatory MFs impairs tissue repair, while persisting tissue reparative MFs, such as in the case of DMD, lead to fibrosis. This indicates that the chronic inflammatory environment specifically impairs the phenotypic transition from pro-inflammatory to pro-resolving/tissue reparative MF subtypes. The factors governing this impaired transition and whether this “halted” inflammatory phenotype can be resolved is still unclear. Furthermore, the exact contribution and roles of each of the different macrophage subtypes, as well as their cellular interactions, remains to be clarified. Recent advances in single-cell analyses and algorithms, along with spatial transcriptomic information, provide potent new strategies to infer cell differentiation trajectories and would greatly aid us in answering some of these questions. In conclusion, the complex immune-microenvironment present in dystrophic muscles leads to disruption of the initiation, progression, and resolution of inflammation, leads to persisted muscle damage, impaired regeneration, and fibrosis, which is linked to muscle dysfunction and the lethal DMD phenotype. Kharraz et al., 2014.

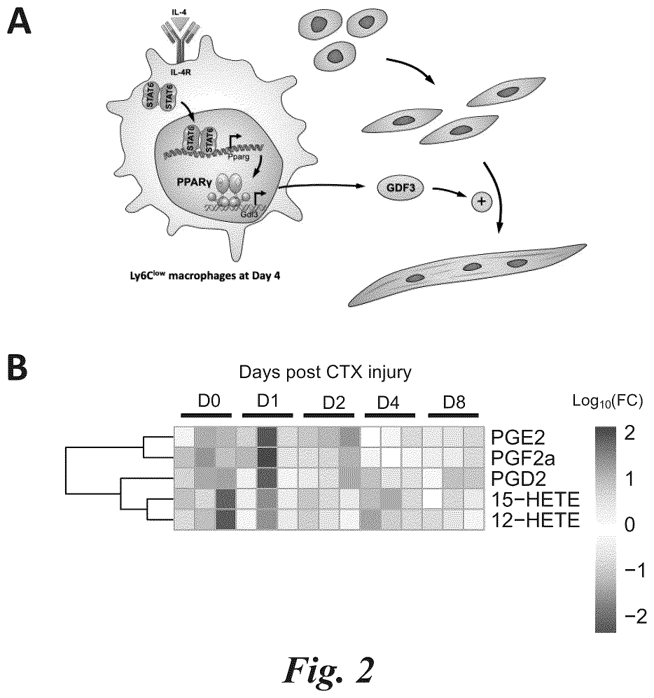

We previously identified a sensory-regulatory-effector axis consisting of STAT6 driving MF polarization, which mediates IL-4 dependent transcriptional events to polarize MFs into the alternative direction. Martinez et al., 2009. As part of this transcriptional program, IL4/STAT6 induces the expression of a ligand-activated transcription factor, PPARγ, Huang et al., 1999, is expressed and activated in repair MFs. Varga et al., 2016a. In turn, PPARγ contributes to IL4/STAT6-dependent transcriptional events. Daniel et al., 2018. One of the genes this receptor regulates is Growth Differentiation Factor 3 (GDF3), a member of the TGFβ family. Levine and Brivanlou, 2006; Lavine et al., 2009. GDF3 is exclusively expressed in MFs in the context of the regenerating muscle and molecularly contributes to myoblast fusion, Varga et al., 2016a. (FIG. 2A).

This discovery serves as proof-of-concept that using unbiased expression profiling and epigenomic approaches coupled with bioinformatics data integration can serve as a starting point to mechanistic investigations to unravel the contribution of immune cells to tissue regeneration. Recently, we took these findings further and tested if GDF3 had effects in vivo on pathological (deficient) cases of skeletal muscle regeneration. We have used aged mice, which have been suggested to have similarities to DMD, Baron et al., 2011, and show a marked delay in regeneration after CTX injury. Patsalos et al., 2018. Using recombinant GDF3 injected into the muscle directly, we were able to alleviate some of the delay, arguing that, at least in principle, GDF3 can be therapeutically relevant in disease settings, as well. Furthermore, our lipidomic analysis revealed that 12-HETE, a known PPARγ ligand is present in injured muscle and can potentially regulate PPARγ activity14 (FIG. 2B).

We have extended this research more recently and using gene expression kinetics-based clustering of blood circulating Ly6CHi, infiltrating inflammatory Ly6C^{High}, and reparative Ly6C^{Low} macrophages, isolated from injured muscle, identified the TGF-β superfamily member, GDF-15, as a component of the regeneration promoting gene expression program. Myeloid GDF-15 is required for proper muscle regeneration following acute sterile injury, as revealed by gain- and loss-of-function studies. Mechanistically, GDF-15 acts both on proliferating myoblasts and on muscle-infiltrating myeloid cells. Epigenomic analyses of upstream regulators of Gdf15 expression identified that it is under the control of nuclear receptors RXR/PPARγ.

Finally, immune single-cell RNA-seq profiling revealed that GDF15 is co-expressed with other known muscle regeneration-associated growth factors, and their expression is limited to a unique subpopulation of repair-type macrophages (growth factor-expressing macrophages [GFEMs]. Patsalos et al., 2022. Very recently, PPARγ agonists have been shown to promote the resolution of myelofibrosis in mice. Lambert et al., 2021. These data make a compelling case for and collectively point to PPARγ as an exploitable target to promote the pro-regenerative activity of macrophages.

### 1.2 Scope

We have been seeking to understand the molecular mechanisms underlying this MF phenotype transition for the purpose of identifying immune-targeting therapeutics for muscle diseases. As part of this effort, we have discovered that activation of the transcription factor, peroxisome proliferator-activated receptor (PPAR)γ contributes to the transition of MFs to a pro-regenerative phenotype and is required for the efficient and complete regeneration of muscle following injury.

The presently disclosed subject matter is directed to evaluating pharmacological activation of PPARγ as a treatment strategy for dystrophic muscle. More particularly, the presently disclosed subject matter includes evaluating the target engagement and efficacy of the thiazolidinedione (TZD) class of drugs, including pioglitazone and rosiglitazone, to determine whether pharmacological activation of MF PPARγ will promote a pro-regenerative MF phenotype and restore the regenerative capacity of dystrophic muscle, thereby slowing disease progression in DMD and BMD. This therapeutic application of PPARγ agonism includes the following.

### 1.3 Pharmacological Targeting of PPARγ by TZDs

A motivation for evaluating PPARγ as a target to treat dystrophic muscle is that this strategy can be quickly evaluated in DMD patients, as multiple clinical PPARγ agonists are available. Arnold et al., 2019. In this example, we will evaluate the PPARγ agonists pioglitazone and rosiglitazone, a thiazolidinedione (TZD)-class of drugs previously approved for the treatment of type 2 diabetes.

The anti-diabetic effects of TZDs were discovered in 1982 at Takeda Pharmaceuticals by the demonstration that compounds made to improve upon the lipid-lowering activity of the clofibrate class of compounds, Sohda et al., 1982, if modified on the acidic thiazolidine-2,4-dione moiety, can improve both hyperglycemic and hyperlipidemic clinical characteristics in yellow KK mice. Sohda et al., 1982a; Sohda et al., 1982b; Sohda et al., 1982c.

Subsequently, the first TZD drug, ciglitazone, was shown to reduce blood glucose levels without increasing insulin secretion in KKAy mice and also to reduce peripheral insulin resistance. Fujita et al., 1982. These findings initiated a search for more potent compounds and ultimately resulted in the FDA approval of three drugs as medicines: troglitazone, rosiglitazone, and pioglitazone. Thus, the systematic evolution of ciglitazone led to the discovery of TZDs, a novel and unique class of antidiabetics that improved hyperglycemia and hyperlipidemia without impacting the secretion of insulin from the pancreas. TZDs have been linked to adipocyte differentiation and gene regulation by PPARγ by several groups in the mid-90s. Kletzien et al., 1992a; Kletzien et al., 1992b; Harris et al., 1994; Tontonoz et al., 1994a; Tontonoz et al., 1994b; Lehmann et al., 1995.

Since then, it has been firmly established that the pharmacophore of TZDs is the nuclear receptor PPARγ. The two currently commercially available, FDA-approved medicines, rosiglitazone, and pioglitazone, have distinct pharmacological profiles, though. Rosiglitazone is a selective PPARγ agonist, whereas pioglitazone has weak PPARα agonist activity, which manifests in clinically beneficial lipid-lowering activity. Sakamoto et al., 2000; Betteridge, 2007.

In the early 2000s, pioglitazone and rosiglitazone were among the most prescribed anti-diabetic medications. There have been increasing concerns, however, about their side effects: increased fluid retention, increased incidence of heart failure, weight gain, increased bladder cancer, and peripheral fractures. Shah and Mudaliar, 2010; Lebovitz, 2019. These issues appear to be greatly exaggerated. Clinical studies have examined the effects of pioglitazone and rosiglitazone on these various outcomes and found that cardiovascular toxicity with rosiglitazone and the increase in bladder cancer are no longer considered significant issues. Lebovitz, 2019; Devchand et al., 2018. There also are new data to show that pioglitazone treatment reduces myocardial infarction and ischemic strokes. Dormandy et al., 2005. Recent preclinical data indicate that pioglitazone treatment also reduces pulmonary hypertension, a cause of right ventricle failure. Legchenko et al., 2018.

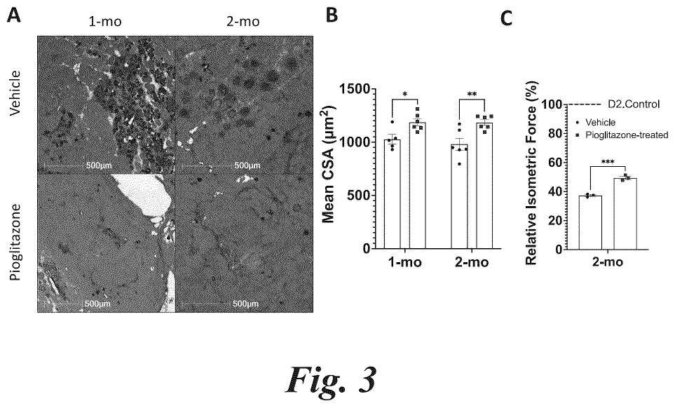

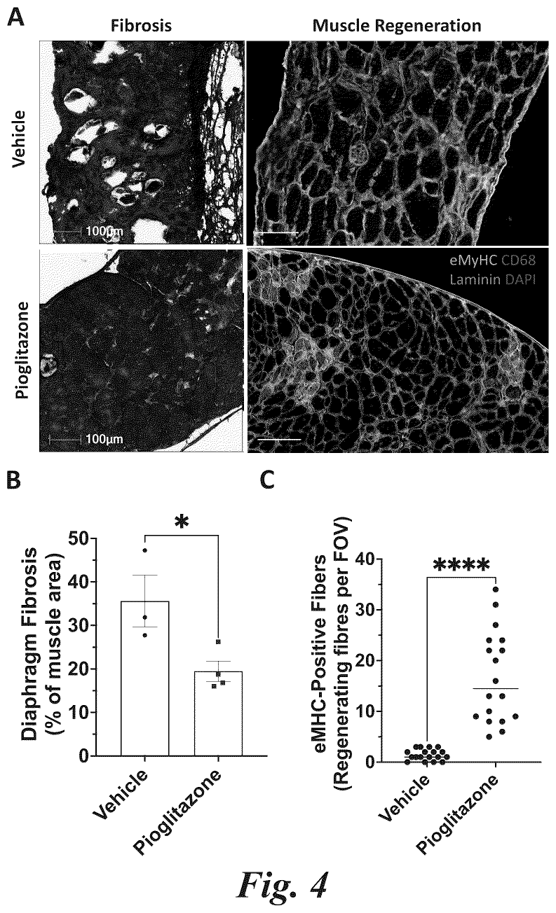

Side-effect profiles of TZDs revisited: Although TZDs have been linked to possible cardiovascular events resulting from off-target effects, this does not appear to be the case for pioglitazone. Recent evidence suggests pioglitazone's safety profile is better than other drugs of this class, Soccio et al., 2014, and patients receiving pioglitazone exhibit improved cardiac function. Clarke et al., 2017. Furthermore, pioglitazone also has shown efficacy in preventing pulmonary hypertension, Legchenko et al., 2018, an additional concern amongst DMD patients. Yotsukura et al., 1988. New data concerning TZD-mediated edema, congestive heart failure, and bone fractures improves the clinician's ability to select and prescribe the one with minimal side effects. Lebovitz, 2019; Devchand et al., 2018; Clarke et al., 2017. Collectively these new findings and the fact that TZDs are now generic make them an attractive target for increased use and for investigations for additional mechanisms of action to repurpose these drugs to contribute to unmet clinical needs in complex diseases. Lebovitz, 2019. Nonetheless, our compelling preliminary data, the successful demonstration that pioglitazone positively affects dystrophic muscle phenotype (FIG. 3 and FIG. 4) also will set the stage for future evaluations of emerging PPARγ agonists with fewer off-target effects. Soccio et al., 2014. In addition, this premise is supported by the favorable metabolic effects of TZDs on whole-body metabolism. For example, findings support the notion that TZD treatment causes increased FA uptake and TG accumulation in skeletal muscle under insulin-stimulated conditions. Todd et al., 2007. TZDs also suppress the inflammatory response to dietary lipid overload, which is a mechanism that correlates strongly with insulin sensitivity. Todd et al., 2007. Activation of PPARγ leads to peripheral insulin sensitization, while PPARγ activation leads to increased fatty acid oxidation and lowering of circulating lipid levels. This latter activity is relevant in the case of pioglitazone, which is a strong PPARγ and a weak PPARα agonist. Anti-inflammatory effects of TZDs, in particular rosiglitazone's also are well documented. Chawla et al., 2001; Ricote et al., 1998. These overlapping, but distinct pharmacological and clinical profiles provide the rationale for the comparative studies proposed here.

### 1.3.1 Evaluate and Compare the Target Engagement of Two PPARγ Agonists in Mouse Models of DMD and BMD

Without wishing to be bound to any one particular theory, it is thought that activation of MF PPARγ in dystrophic muscle using synthetic agonists (e.g., thiazolidinediones, such as pioglitazone and rosiglitazone) regulates MF phenotype transition and promotes pro-regenerative gene expression profiles, resulting in amelioration of disease progression.

Two doses of each of the PPARγ agonists, e.g., pioglitazone or rosiglitazone, will be tested in mouse cohorts representative of early- and late-stage disease progression. The effects of these dosing regimens on other tissues will be assessed by gene expression and by assays for cardiovascular, respiratory, renal, osteal, and hepatic effects. The impact of these compounds on muscle disease will be assessed by measuring in vivo and ex vivo muscle function and body composition.

### 1.3.2 Test the Efficacy of PPARγ Agonism as a Muscle Therapeutic in Mouse Models of DMD and BMD

Again, without wishing to be bound to any one particular theory, it is thought that targeting MF PPARγ will promote beneficial remodeling of skeletal muscle at all disease stages in mice that model DMD and BMD and will improve their cardiopulmonary function. This example will evaluate the efficacy of long-term PPARγ agonism for the treatment of skeletal muscle disease in mice that model DMD and BMD. A thiazolidinedione, such as pioglitazone and rosiglitazone, will be administered to sedentary and wheel-running mice representative of multiple stages of disease progression. Efficacy will be determined through measures of limb and respiratory muscle function and histopathology. Echocardiography and cardiac histology will be implemented to evaluate the impact of PPARγ agonism on the dystrophic heart disease progression.

### 1.3.3 Dose Considerations

The recommended human doses for ACTOS® (pioglitazone) are 15-45 mg per day with congenital heart disease 15 mg and with close monitoring 45 mg, for AVANDIA® (rosiglitazone) 2-8 mg per day. Based on these, we determined the equivalent mouse dose ranges for pioglitazone 10-60 mg/kg/day for rosiglitazone 2-8 mg/kg/day. We will use two pioglitazone doses of 10 mg/kg and 50 mg/kg and two doses for rosiglitazone, 2 mg/kg and 8 mg/kg, which is roughly equivalent to the low and high end of the range of the human dose (normalized to the body surface area) that has shown improved heart function in diabetic patients40 and has demonstrated pro-regenerative effects in preliminary studies in aged D2.mdx mice (FIG. 4).

A goal is to systematically evaluate the use of TZDs as a means to promote muscle regeneration and improve dystrophic muscle as monotherapy for DMD and BMD. This strategy also has potential utility in combination with gene therapy.

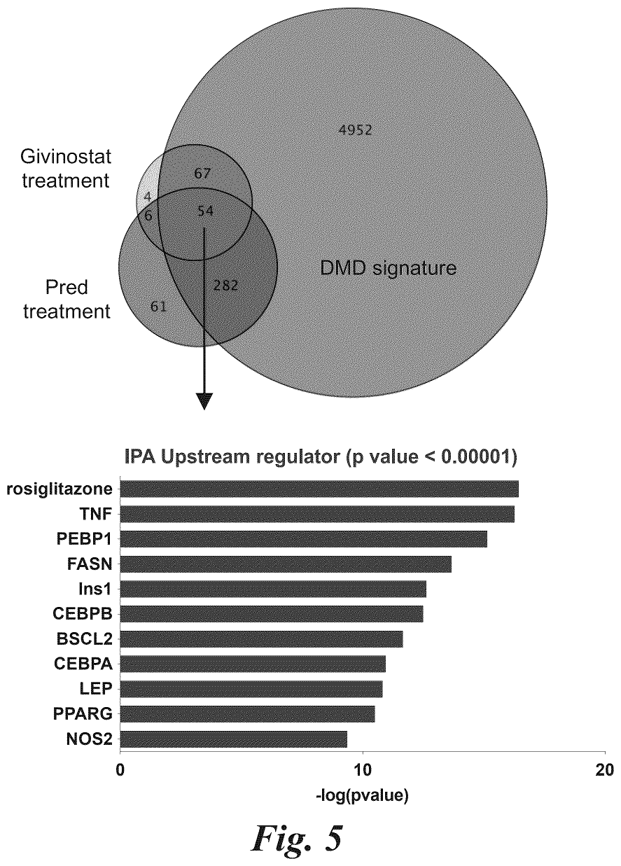

Without wishing to be bound to any one particular theory, it is thought that TZDs activate MF PPARγ, which in turn will bias the milieu of severely dystrophic muscle towards regeneration. This hypothesis is based on the following lines of evidence: (1) Dystrophic disease progression exhibits distinct stages differing in the degree of immune infiltration, muscle degeneration, and fibrotic progression (FIG. 1). (2) PPARγ is required for repair MF function and promotion of muscle regeneration (FIG. 2A). (3) Gene expression signature characteristic of PPARγ activation is present in pharmacologically treated dystrophic muscle and correlates with therapeutic efficacy (FIG. 5). (4) Dosing D2.mdx mice with pioglitazone improves muscle structure and function and reduces fibrosis even when applied at late stages (8 months) (FIG. 3 and FIG. 4).

In performing the experiments proposed in this example, we aim to accomplish: (1) Dissecting the contribution of PPARγ activated by two different TZDs to the immune infiltrate, the single-cell and spatial transcriptome in whole muscle and isolated immune cell populations during disease progression. (2) To compare the impact of PPARγ activation by pioglitazone or rosiglitazone on DMD disease progression. (3) Implementation of a novel therapeutic strategy by engaging early (inflammatory) or late (repair) immune targets by using the more efficacious PPARγ agonist in DMD and along with transgenic micro-dystrophin that models BMD.

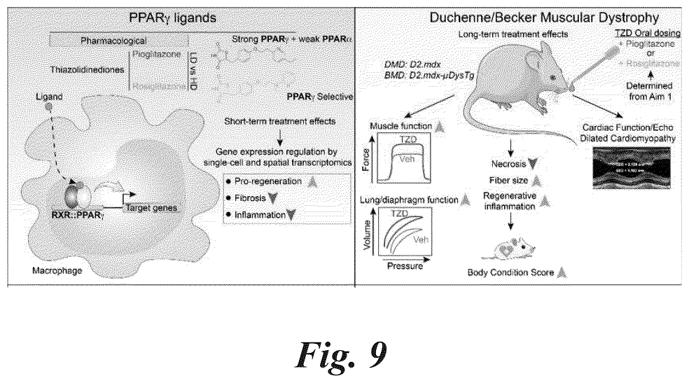

The contribution of this example will be a comparative analysis of two FDA-approved synthetic PPARγ ligands' effect on the gene regulation, the immune infiltrates, and disease progression of the D2.mdx mouse model of DMD and of the micro-dystrophin expressing model of BMD. This contribution is significant because it will repurpose TZDs as novel therapeutics in dystrophies and open new avenues of therapeutic interventions not only in DMD and BMD but also in other diseases characterized by progressive fibrosis, including limb-girdle muscular dystrophies (FIG. 9).

There are multiple conceptual and technical innovative aspects in this example including, but not limited to: (1) systematic comparative in vivo analyses of two doses of two TZDs on immune cell subpopulations in DMD and BMD disease progression at an unprecedented granularity (single-cell transcriptomics) and tissue level resolution of cellular interaction using spatial transcriptomics; (2) validating MF PPARγ as a bivalent (anti-inflammatory and pro-regeneration) therapeutic target in DMD and BMD and determining the compound and the dose with the highest efficacy and minimal side effects; (3) implementation of a novel therapeutic regimen that can benefit both DMD and BMD, targeting failed regeneration in muscles, using PPARγ activators to engage the immune component of DMD and improve the muscle phenotype associated with the disease. Observed efficacy in the BMD model may indicate utility as an adjunct therapy for micro-dystrophin gene therapy; and (4) integrated use of single-cell, spatial transcriptomics during DMD progression and upon PPARγ ligand treatment to discover novel cellular states and tissue level interactions.

### 1.3.4 Approach

### 1.3.4.1 Modeling DMD and BMD

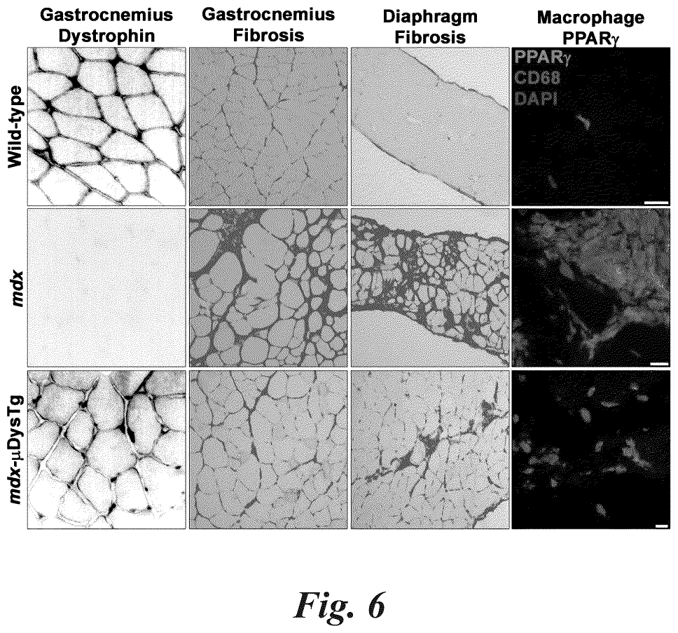

DMD will be modeled using the D2.mdx mouse, which harbors the dystrophin-null mdx allele on the DBA/2J genetic background. This model better recapitulates several aspects of the human disease than C57-based mdx mice, including progressive fibrosis, muscle wasting, weakness, and regenerative impairments. Hammers et al., 2020. To model BMD skeletal muscle, we have crossed a micro-dystrophin transgene (μDysTg) onto the D2.mdx background. The AR4-R23ACT version of micro-dystrophin is driven by the human skeletal actin (HSA) promoter and expressed in the skeletal muscles, but not the heart, of these mice. Li et al., 2011. This genetically encoded dystrophin truncation is identical in structure to a micro-dystrophin version used in an ongoing gene therapy clinical trial, Mendell et al., 2020, and only partially rescues the skeletal muscle disease of D2.mdx mice as evident by the pathological fibrosis found in the limb muscles and diaphragms of these mice (FIG. 6). Importantly, intramuscular MFs (identified by CD68 labeling) of both the DMD and BMD models exhibit PPARγ staining in the nucleus, whereas resident MFs of wild-type muscles do not (FIG. 6).

### 1.3.4.1.1 Evaluate and Compare the Target Engagement of Two PPARγ Agonists in Mouse Models of DMD and BMD

PPARγ is expressed in a subset of muscle-infiltrating MFs. Activation of MF PPARγ by synthetic activators (pioglitazone or rosiglitazone) results in overlapping but distinct, dose-dependent gene expression changes, regulation of MF phenotype transition, effector function, and skeletal muscle and heart gene regulation.

### 1.3.4.1.1A Identify and Characterize the Consequence of Pharmacological Activation of MF PPARγ on Immune Infiltrate, Gene Regulation, and Disease Progression at Early Stages in DMD

We will investigate MF populations and gene expression profiles in dystrophic muscle (DMD) and dystrophic muscle corrected by micro-dystrophin expressing transgene (BMD model) using flow cytometry, immune cell profiling, and innovative (single-cell and spatial) transcriptomic analyses integrated by bioinformatics upon pharmacological activation of PPARγ by pioglitazone or rosiglitazone in two different doses (high and low) representing the dose range used in humans.

Immune cell infiltration in muscle injury is a complex process involving the recruitment of a subtype of circulating Ly6C^{High} monocytes. Geissmann et al., 2003; Varga et al., 2013. These cells then convert to inflammatory MFs (still Ly6C^{High}) and later to repair type ones (Ly6C^{Low}). Chazaud, 2014. It also has been shown that inflammatory monocytes promote DMD pathology. Mojumdar et al., 2014. In light of these facts, a more thorough and in-depth evaluation of immune cells their subtype specification is warranted. To critically evaluate their roles, one needs to characterize them by studying their global, single-cell, and spatial transcriptional gene expression profile. MF infiltration into inflamed tissues has been implicated in chronic inflammation-induced organ fibrosis. These MFs are derived from CCR2⁺ inflammatory monocytes or Ly6C^{High} monocytes. The general concept is that prolonged inflammation induces a shift from the Th1 or M1 phenotype to the Th2 or M2 phenotype over several weeks or months. Barton et al., 2010. The resulting Th2 phenotype and the production of Th2 cytokines such as IL4, IL13, and IL10 induce the infiltration of pro-fibrotic eosinophils via cognate (i.e., eotaxin) production. Therefore, there is an emerging conserved sequence of events characteristic of fibrotic responses in several tissues involving inflammatory monocyte/macrophage invasion into the injured tissue. This process is driven by the changing milieu, including the mediator lipidome and the changing immune phenotype of the cells present.

We have systematically profiled and identified regulatory and effector mechanisms of muscle MFs using a sterile muscle injury model in mice injured by cardiotoxin (CTX). The CTX model is widely used to assess the capacity of skeletal muscle to quickly and completely regenerate. Hardy et al., 2016. During these studies, we evaluated the contribution of Ly6C^{High} (inflammatory) and Ly6C^{Low} (patrolling), Geissmann et al., 2003, circulating monocytes to infiltrating muscle MFs and found that inflammatory monocytes infiltrate the injured tissue first, and these convert to repair type MFs later in situ. Varga et al., 2013. This is an important concept because it argues that inflammatory (Ly6C^{High}) MFs can be driven or re-programmed to become repair type (Ly6C^{Low}) cells. Arnold et al., 2007; Varga et al., 2016b. Next, we generated a coherent and informative set of expression profiles on muscle inflammatory and repair MFs from the CTX sterile injury model using cell sorting coupled to global expression profiling in a time course experiment. Varga et al., 2016b. These results show the highly dynamic nature of the muscle MF response at the molecular level and document that a specific signature, primarily driven by the cellular milieu, characterizes inflammatory and repair MFs at each step of tissue injury and repair. Varga et al., 2016b. This work and datasets were later complemented with RNA-seq analyses of the same cell populations allowing us to validate and extend the findings reported earlier. Varga et al., 2016a.

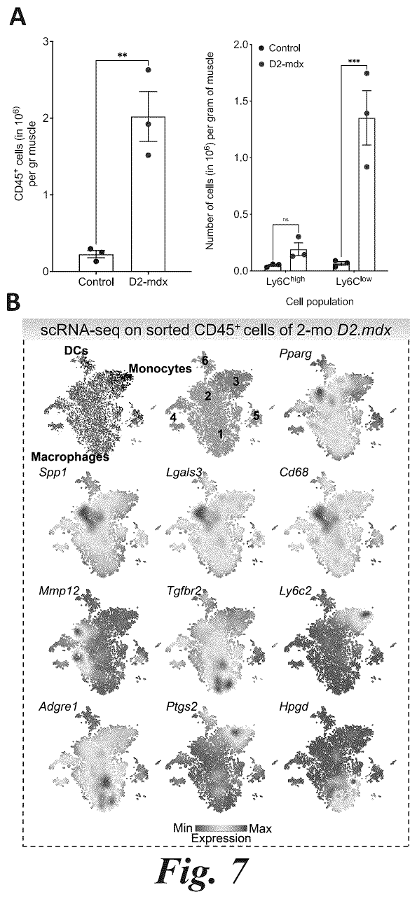

In this example, we are proposing to determine and characterize the impact of a range of doses of two TZDs acting on PPARγ in DMD and BMD progression by measuring target engagement, efficacy, and side effects. Currently, there are no specific immune targeting therapeutic agents available with bivalent effects in suppressing inflammation and promoting functional muscle recovery. Therefore, new approaches are needed that improve muscle function while decreasing inflammation and fibrosis. The presented analyses suggest that MFs are present in the D2.mdx dystrophic muscle at significant numbers (FIG. 7A), and single-cell RNA-seq reveals that they express genes contributing to mediator lipid production (FIG. 7B) (Ptgs2, Hpgds) and fatty acid metabolism, including lipid sensing transcription factors such as PPARγ (FIG. 7B).

We will determine the contribution of TZD mediated PPARγ activation to muscle immune infiltrates, MF phenotypes and determine the dynamically changing cellular composition of immune cells at the single-cell and spatial level. These will allow us to point to the molecular mechanisms driving MF immune function during muscular dystrophy and help us to identify new markers for MF subsets to refine the characterization of these cell subpopulations in the DMD and BMD settings. In addition, kinetic analyses will decipher the interactome of MFs with their neighboring cells, as this landscape constantly evolves during the dystrophy-driven inflammatory response. In this context, it has been recently speculated that tissue-resident MFs may play a role in modulating inflammation by recruiting monocytes to damaged muscle according to the degree of injury or disease progression. De Micheli et al., 2020; Giordani et al., 2019.

Our preliminary data using single-cell RNA-seq from CD45⁺ cells isolated from dystrophic muscles (2-mo D2.mdx) reveals cell populations that are present specifically either at early (inflammation) or late stages of the normal muscle repair process following acute sterile injury. First, we observed APCs characterized by the expression of MHC class II proteins such as Cd74 and the H2 family. Second, we identified pro-inflammatory MFs that express Cc19 (a chemokine that attracts Cd11b⁺Ccr1⁺ dendritic cells), Ccr2 (a chemokine involved in monocyte chemotaxis), and Ly6c2. Third, we observed a less well-defined group of cells that express a wide variety of markers, including Cd68+ monocytes. Baitsch et al., 2011; Ho et al., 2016. Finally, we detected some Aif1⁺ MFs and/or dendritic cells that have been reported to modulate muscle repair. Borneman et al., 2000. MFs from dystrophic muscle appear to be predominantly repair-type ones with a minor population of inflammatory ones. Significantly, the majority of the repair MFs in the DMD sample express PPARγ (FIG. 7B). It remains to be resolved, however, how regulatory factors like PPARγ and surface markers define this monocytic/MF continuum and where cells from DMD and BMD fall in this spectrum and impact muscle function.

We also will determine how the chronic inflammatory state of DMD and BMD impacts immune cell composition. Thus, this example also is designed to be a large-scale discovery screen for additional therapeutic targets for future development. The following approaches will be used (a) histology and flow cytometry to characterize and quantitate disease progression and CD45⁺ myeloid cells, (b) bulk, (c) single cell, and (d) histology coupled spatial transcriptomic analysis in models of disease progression in ten treatment groups: five in DMD models (1) in normal/unperturbed disease progression with vehicle treatment and (2-3) in the presence of an orally delivered FDA approved PPARγ agonist pioglitazone at doses 10 or 50 mg/kg/day or (4-5) in the presence two doses of rosiglitazone 2 or 8 mg/kg/day) and another five in the micro-dystrophy overexpressing milder BMD model. The disease progression, immune cell composition, gene expression, and transcriptomic changes will be determined, integrated, and compared to assess optimal target engagement efficacy and to determine the more suitable compound and dose.

### 1.3.4.1.1A-i Experimental Design and Methods

Ten cohorts (N=5 per condition) of male D2.mdx mice (1-month old) will be treated with vehicle, pioglitazone (10 or 50 mg/kg), or rosiglitazone (2 or 8 mg/kg) daily for four weeks. We will then evaluate DMD or BMD disease progression and determine i) immune cell composition (using flow cytometry), ii) bulk and single-cell, and iii) histology-combined spatial transcriptomics.

### 1.3.4.1.1A-i-a Histology and Flow Cytometry

Muscle histology will be used for the evaluation of DMD ad BMD disease progression and assessing muscle specimens. Routine histochemistry, which is typically performed on frozen tissue, commonly includes H&E and Masson's Trichrome stains. General morphology, including fiber size (cross-sectional area (CSA)), split fibers, location of nuclei, regenerating and degenerating fibers, connective tissue, and inflammatory cells, will be quantified using the HALO digital pathology platform (Indica Labs) for morphometric analysis. Histological assays will be complemented with bulk RNA-seq from total muscle tissue flow cytometry methods for the isolation and characterization of immune cell populations from the muscle of the D2.mdx mouse model of DMD and the micro-dystrophy overexpressing BMD model. We will prepare single-cell suspensions of dystrophic muscle (GAST, QUAD, TA, EDL) that maintain the integrity of cell-surface markers and use CD45+ magnetic bead separation and antibody panels from Biolegend (α-CD11b, α-Ly6C, α-F4/80, α-CD206, α-Ly6G, α-CD11c, α-MHCII: I-A/I-E) for identifying and studying immune cell populations in the skeletal muscle of D2.mdx mice. Samples will be analyzed using a BC CytoFLEX LX, and macrophage populations will be sorted on a BC MoFlo Astrios equipped with a 70-μm nozzle and five lasers (355 nm, 405 nm, 488 nm, 561, 640 nm) for downstream analysis (i.e., scRNA-seq).

### 1.3.4.1.1A-i-b Single-Cell Transcriptomics

We will generate an immune (CD45⁺) single-cell transcriptomic atlas of dystrophic muscle (DMD and BMD) using the animal cohorts to describe above the immune cell/monocytic continuum, and multicellular communication networks from vehicle-and TZD treated samples using the gastrocnemius muscle from the two models. To gather a comprehensive view of these processes, we will use droplet-based single-cell 3′RNA-sequencing (scRNA-seq) on the 10X Chromium platform to collect a multi-cell-type transcriptomic reference time-course at an early time-point initiated at one month of age) and treatments (vehicle and TZDs-treated at two doses each; 4-week long treatment) from dystrophic gastrocnemius muscles (n=5 D2.mdx mice per time-point per treatment). We will use 20,000 cells per condition and generate at least 100,000 reads per cell to saturate the sequencing and get optimal datasets as described. Zhang et al., 2020. For the bioinformatic analysis, we will use the Seurat package for scRNA-seq data filtering and processing. Cell communication signals, acting through secreted ligands binding to receptors on satellite, fibroblasts, and fibro-adipogenic progenitors (FAPs), govern a multitude of cell-fate regulation mechanisms critical for muscle homeostasis and regeneration. Yin et al., 2013. We will generate a model that scores for interactions between receptors expressed by non-immune cells and ligands expressed by monocytes and other mature immune cell types. Skelly et al., 2018.

### 1.3.4.1.1A-i-c Spatial Transcriptomics

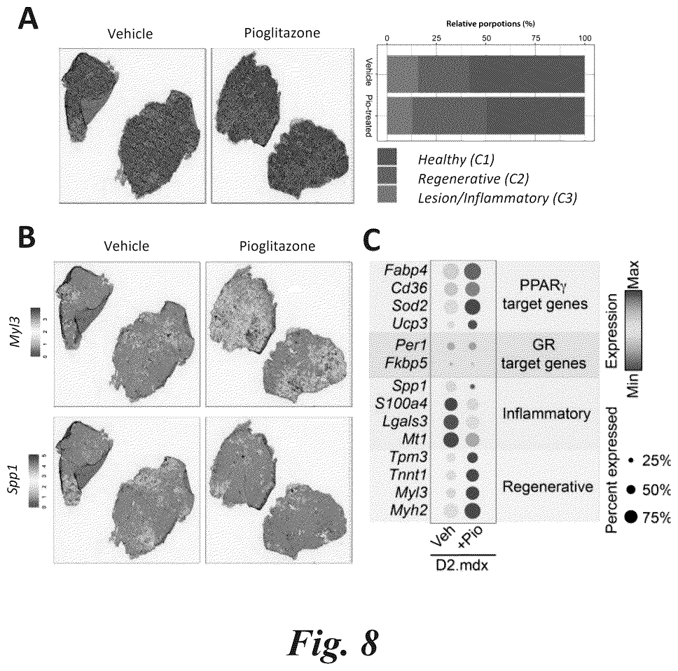

Fibrosis and ectopic adipose tissue deposition occurring in DMD and BMD patients result from deregulation of cellular communication within dystrophic muscle and as a consequence of unresolved cycles of degeneration. The spatiotemporal ordering of molecular events that drive these processes, however, remains poorly understood. Inherent limitations of current gene expression profiling technologies, such as low throughput or lack of spatial resolution, have thus far hindered efforts to understand how such dysfunction contributes to the onset and progression of DMD pathology in various tissues. Given the stereotyped cellular organization of skeletal muscle and the importance of intercellular communication in DMD ad BMD progression, we reasoned that a spatially resolved view of disease-driven gene expression changes would be needed to reveal the relevant subpopulations of cells involved and characterize the underlying molecular mechanisms that trigger and maintain the disease phenotype. Here, we will use spatial transcriptomics (Visium kit by 10X Genomics) in combination with the single-cell RNA-seq approach described above to obtain gene expression measurements of mouse skeletal muscle (gastrocnemius) upon PPARγ activation with the two TZDs, in two doses to distinguish regional differences between immune and myogenic populations at early and late time points of disease progression. Space Ranger (10X Genomics) and stLearn pipelines will be used. Our preliminary data obtained on gastrocnemius muscle of pioglitazone treated 2-mo D2.mdx mice reveal three bioinformatically predicted k-means clusters of gene expression that overlap with the three areas of distinct histological features annotated manually by a pathologist (FIG. 8A). Importantly, we observed increased areas of regeneration (FIG. 8A-FIG. 8C), reduced inflammatory gene expression (FIG. 8A-FIG. 8C), and induction of known PPARγ target genes but not Glucocorticoid Receptor (GR) target genes (FIG. 8C), establishing dynamic changes in gene expression upon ligand treatment and target engagement in the muscle tissue. Thus pioglitazone engages its target, PPARγ, and does not activate GRs.

The acquired data will allow us to evaluate the target engagement and efficacy of the two TZDs and the two doses (low and high) on PPARγ mediated gene expression, immune profiles, and disease progression. We will quantify target engagement by evaluating gene expression changes and compare first the responses to the low and high doses in the case of each compound and then the two compounds and see if we detect a dose-dependent increase in the expression of direct PPARγ targets in macrophages (FABP4, CD36, GDF3, GDF15) in the higher doses or between the two compounds.

We also will look at immune cell profiles and see if there are differences in myeloid cell subtypes and/or immune phenotypes (growth factor production, MHCII expression) measured by flow cytometry. In addition, we will compare vehicle-treated (as normal disease progression) to the two different TZDs and the low and high doses and evaluate the amount of immune infiltrate, fiber size distribution, and new fiber formation.

Based on these assessments, we will determine if pioglitazone or rosiglitazone is superior and if the high dose results in a more efficacious target engagement. Our anticipation is that pioglitazone will induce a broader gene expression change due to its weak PPARα activating effect besides strong PPARγ activation. This evaluation will be complemented by gene expression assessment in skeletal and heart muscle to measure PPARα regulated genes, i.e., the ones associated with fatty acid oxidation (LPL, FABP3, SCD1, PDK4). Crossland et al., 2021. Collectively these analyses will lead to the selection of the compound and the dose for further evaluation. Ideally, we would like to use the compound and the dose with the higher efficacy and most benefit with fewer side effects in terms of disease progression. Our results also will provide a comprehensive analysis with spatial and temporal resolution of the cellular composition (flow cytometry), myeloid cell (single-cell), whole tissue (spatial) gene expression changes during DMD ad BMD progression, and the impact of TZD-mediated gene expression changes on these. This will serve as a map to identify pathways contributing to inflammatory and repair activity that can be targeted in therapies and will establish PPARγ as an activated transcription factor and a key driver of MF phenotypic change and resolution and repurpose TZDs for the treatment of DMD and BMD. Based on our results demonstrating that there is an elevated number of Ly6Clow MFs (FIG. 7A) in D2.mdx mice, we expect that restoring or promoting resolution by PPARγ activation will rescue or alleviate this defect, reduce fibrosis, and enhance muscle regeneration. One caveat to this approach is that systemically activating PPARγ may have other effects on metabolism and thereby alter tissue inflammation. This can be addressed using tissue-specific knock-out models to gain mechanistic insight. This is beyond the scope of this study aimed at repurposing TZDs. Analyzing single nuclei rather than single cells is an important alternative strategy, which addresses issues with tissues that cannot be readily dissociated into a single-cell suspension. van den Brink et al., 2017. From a bioinformatics point of view, we do not expect any difficulties in analyzing the datasets. The results of these efforts will be used to generate a searchable online database for the DMD and BMD community, which we believe will hasten therapeutic development from identified targets. This database will be invaluable to identify direct PPARγ targets, including secreted ones, new biomarkers, and immune targets to be pursued in future studies.

### 1.3.4.1.1B Compare the Efficacy and Safety of Pioglitazone or Rosiglitazone Treatment of DMD and BMD Starting at the Later Fibrotic Stages of the Disease (8 Months)

Treatment of late-stage DMD mice with pioglitazone or rosiglitazone results in new fiber formation and has significant clinical benefits, slows down disease progression with minimal but distinct side effect profiles. Following the identification of a population of PPARγ-expressing MFs in dystrophic muscle (FIG. 6 and FIG. 7) and the convergence of PPARγ activation pathways in muscle gene expression profiles following treatments that improve diaphragm function (FIG. 5), we performed a pilot trial for pioglitazone in older D2.mdx mice, which exhibit severe muscle decrements representative of DMD muscle pathology (FIG. 1). Hammers et al., 2020.

The short-term (4 weeks) treatment of 8 month-old D2.mdx with 10 mg/kg pioglitazone (or vehicle control) resulted in significant decreases in diaphragm fibrosis and an increase in embryonic myosin heavy chain (eMyHC) staining (FIG. 4), an indicator of activated muscle regeneration. The positive results of this pilot study will be followed up in this example, where the short-term benefits and tissue-specific gene expression profiles resulting from pioglitazone or rosiglitazone treatment will be determined to identify the TZD compound/dose that better conveys muscle benefits in both the DMD and BMD models. Gene expression and histological changes associated with disease progression (FIG. 1), cardiovascular, kidney, bone, and liver parameters will be assessed using two different doses (low and high end within the used human range) of the two compounds. The clinical benefit and the efficacy and tissue-specific profile of pioglitazone or rosiglitazone treatment will be determined at a late time point of 8 months with a short treatment (4 weeks). Gene expression changes, disease progression, cardiovascular, kidney, bone, and liver parameters will be assessed using two different doses (low and high end of used human range) of the two compounds.

### 1.3.4.1.1B-i Experimental Design and Methods

Ten cohorts (N=5 per condition, five per strain) of male D2.mdx mice (8-month old) and D2.mdx-μDysTg will be treated with vehicle, pioglitazone (10 or 50 mg/kg) or rosiglitazone (2 or 8 mg/kg) daily for four weeks. We will then evaluate DMD or BMD disease progression using histology and morphometry and the primary endpoints listed in Table 1. In addition, spatial transcriptomics to assess cellular gene expression changes will be used as described above.

TABLE 1
Primary endpoint measure (UF and JHU)

| Functional | In vivo and Ex vivo muscle function | | Forelimb grip strength, Hanging test | | |
|---|---|---|---|---|---|
| Biochemical | Serum Creatine | Tissue | Membrane permeability | | |
| | Kinase | Hydroxyproline | (Evans Blue uptake as a measure of | | |
| | (muscle damage) | (Fibrosis) | muscle fibers damage) | | |
| Morphological | Bodyweight | Muscle mass | Organ mass | Tibia | In vivo |
| | | | | length | imaging - |
| | | | | | NMR, μCT |
| | | | | | (lean mass, |
| | | | | | bone |
| | | | | | density) |
| Histological | Muscle | Muscle fibrosis | Muscle fiber size | | eMyHC |
| | morphology | (Trichrome, | (Cross sectional area) | | staining for |
| | (H&E) | Picrosirius Red) | | | nascent |
| | | | | | fibers |

The TZD doses chosen in our studies have been shown to be well tolerated and were evaluated in repeat-dose studies in humans, often in parallel with rodent studies, where more extensive evaluations were made in a regulatory compliant manner, including gross measures of effect such as body weights and clinical observations, microscopic measures based on histological evaluation of tissues, and evaluation of clinical chemistry and hematology parameters. Toxicity, however, may still be observed in these repeat-dose studies, and thus we will address any potential development-limiting toxicity by investigating the status of the liver, lungs, and kidney as well as any external signs of toxicity, such as changes in the skin, eyes, and mucous membranes, respiration, behavioral patterns, salivation, diarrhea, or tremors.

Blood will be used to evaluate hematological, biochemical, and toxicological parameters. Hematological analysis using an automated counter will be performed using total blood collected with EDTA (IDEXX BioAnalytics). The evaluated parameters will be the numbers of red blood cells, platelets, leukocytes, band cells, lymphocytes, monocytes, and eosinophils, and the amount of hemoglobin, hematocrit, mean corpuscular volume, mean corpuscular hemoglobin, and plasma proteins. Serum from the blood samples also will be separated for further analysis. Biochemical parameters (plasma glucose, lipid/cholesterol/triglycerides levels, markers of kidney and renal toxicity; levels of urea, creatinine, ALT, AST) will be determined by enzymatic assays, using services (IDEXX BioAnalytics) and diagnostic kits following the manufacturers' instructions (Dimension AR, Delaware). Bai et al., 2018. To assess renal tubule damage from DA, urinary markers of tubule injury, neutrophil gelatinase-associated lipocalin (uNGAL), and kidney injury molecule-1 (uKIM-1) also will be examined. Funk et al., 2014. Finally, combining μCT (Rigaku Quantum FX) and FlexiVent (SCIREQ), Artaechevarria et al., 2010; De Vleeschauwer et al., 2011, will be used monthly to evaluate and monitor in vivo bone density and pulmonary function (see also Vertebrate animals section), respectively.

This example also will include cohorts of mice receiving identical TZD treatments for the purpose of comparing the functional impact of these compounds. In this evaluation (performed at UF), both DMD and BMD models will begin TZD treatments at either 1 (models early-stage DMD) or 8 (models late-stage DMD) months of age (n=12 for all groups). Those beginning at 1 month will reach terminal endpoint after 3 months of treatment (4 months of age), whereas those initiated at 8 months will be treated for 4 months prior to terminal endpoints (12 months of age) in order to allow more time for the remodeling of severely disease-burdened muscle exhibited by the DMD model. At the designated endpoints, mice will undergo echocardiography to assess cardiac function, grip strength measurements, and ex vivo muscle mechanics to evaluate muscle force production of the diaphragm and EDL muscles. Muscles isolated from these groups will be used for histological and biochemical assessments for markers of inflammation, regeneration, and fibrosis by investigators at both UF and JHU. Age- and treatment-matched D2.WT mice also will be included in this study (n=12). Data obtained from these measurements, in combination with those from the target engagement and safety profiling assays described above, will be used to determine the optimal TZD compound and dose to be investigated as a long-term therapeutic for dystrophic muscle in Aim 2 of this project.

Based on the compelling preliminary data (FIG. 3 and FIG. 4), we anticipate slower disease progression, enhanced regenerative MF activity, improved muscle structure, functional improvements in regeneration, and inflammatory response with both pioglitazone and rosiglitazone treatments. Given pioglitazone's weak PPARα activating ability, a slightly better clinical outcome is anticipated due to beneficial impacts on lipids. Since oral dosing by TZDs is likely to impact other tissues, the global effects on metabolic tissues will be considered by looking at adiposity, muscle mass, glucose levels, and insulin sensitivity.

We also anticipate that neither compound will have significant side effects, and pioglitazone may have an overall larger benefit due to its weak PPARα activity and thus lipid-lowering effect. A genetic approach using MF-specific inducible PPARγ deletion also will be considered in future studies in which the receptor's expression can be turned on and off at will, such as by using the tamoxifen-inducible system. Kallunki et al., 2019. All listed methods are established in the JHU and/or UF laboratories.

### 1.3.4.2 Test the Efficacy of PPARγ Agonism as a Muscle Therapeutic in Mouse Models of DMD and BMD

Targeting MF PPARγ is expected to promote beneficial remodeling of skeletal muscle at all disease stages in mice that model DMD and BMD. Small molecule therapeutics capable of improving the disease state of dystrophic muscle are a major unmet clinical need. This is particularly true for older patients, where a substantial amount of musculature has degenerated and been replaced with fibrosis. We have strong preliminary data that suggest TZD treatment improves dystrophic muscle when initiated at either early (FIG. 3) or late (FIG. 4) stages of DMD disease progression. The purpose of this example is to rigorously evaluate the long-term efficacy of TZDs as a therapeutic for the muscle disease associated with DMD and BMD.

### 1.3.4.2.1 Experimental Design and Methods

This long-term therapeutic trial will consist of DMD and BMD mice receiving vehicle or TZD treatments (compound and dose determined hereinabove) that begin at either 1 or 8 months of age. These initiation-age cohorts will be divided into sedentary and wheel-running groups (n=12). Mice of this experiment will reach the terminal endpoint at 18 months of age. Age-, treatment-, and activity-matched D2.WT mice also will be included in this study (n=12). During the course of this treatment regimen, echocardiography, grip strength, and body composition measurements will be assessed bimonthly and at the endpoint. Serum samples also will be collected at these time points to measure circulating creatine kinase and inflammatory cytokine levels. Wheel running distance, velocity, and duration will be recorded daily using the VitalView Activity Monitoring System (STARR Life Sciences). At the terminal endpoint, in vivo and ex vivo function of the diaphragm and limb muscles will be assessed, and tissues will be collected and prepared for histological and biochemical analyses, including assays for muscle regeneration, fibrosis, and inflammation. Additional assays will investigate satellite cell numbers and ex vivo proliferation and differentiation potential, as well as macrophage cellular profiles. One of the primary causes of death in DMD patients is cardiac and respiratory insufficiencies due to the weakening of the respective muscles. Raman et al., 2015; Politano et al., 1996; Grady et al., 1997; Ponnusamy et al., 2017.

Since TZDs improved muscle function, we expected it to strengthen the cardiac muscle, thereby reversing most, if not all, cardiac pathology. D2.mdx and D2.mdx-μDysTg mice (n=6/group) will be assessed following treatment with vehicle or TZD for the timepoints described above. As already mentioned, an echocardiogram will be performed to determine the architecture and function of the hearts and compare with reference values for age-matched D2.wild-type mice. Furthermore, since diaphragm fibrosis (typically associated with inspiratory muscle weakness and chest wall restriction of lung expansion) was reduced in Pio-treated animals (FIG. 4B-FIG. 4C), and to determine if this effect leads to improvement in lung function as a result of the retained strength of the diaphragm, we will perform a ventilator study with FlexiVent (SCIREQ, Montreal, PQ, Canada) in D2.mdx and D2.mdx-μDysTg mice treated with vehicle or TZD. The parameters of the single-compartment model, Bates and Irvin, 1985, resistance (R) and compliance (C), and the constant phase model, airway resistance (Raw), inertance (I), tissue damping (G), and tissue elastance (H), will be obtained. Total lung capacity (TLC) maneuvers also will be performed between acquisitions. For assessing the contribution of diaphragmatic fibrosis, we will be looking at respiratory factors of stiffness: lung compliance (Crs) and Pressure-Volume loops, tissue-level constriction (G), and peak inspiratory/expiratory flow (PIF/PEF) to reflect inspiratory muscle strength (namely diaphragm) and expiratory muscle strength (internal intercostal and abdominal muscles), respectively. The results from these experiments will provide evidence if PPARγ agonists can also improve the cardiopulmonary function in the DMD/BMD mouse models.

We anticipate that TZD treatment will benefit both the DMD and BMD models, where treatment initiation at the early stage of disease will greatly attenuate muscle fibrosis and allow successful regeneration, helping to preserve muscle function. We anticipate that late-stage treatment initiation will promote beneficial remodeling of disease-burdened muscles, thereby improving and stabilizing muscle function.

Furthermore, we anticipate TZDs will increase the ad libitum wheel running, grip strength, and lean body mass composition during the course of this longitudinal study. Regarding the pulmonary dysfunction, we expect it will be improved by TZD treatment and restore the ejection fraction (EF) to that of normal healthy adult mice (between 55 and 65%). Vinhas et al., 2013. We also expect that mice treated with TZDs will demonstrate an increase in peak inspiratory flow (PIF; reflects inspiratory diaphragm muscle strength) and peak expiratory flow (PEF; expiratory muscle strength of internal intercostal and abdominal muscles) and a significant decrease in airway resistance to 50 mg/ml methacholine (MeCh) compared with mice receiving vehicle. These studies will provide a strong foundation to initiate additional preclinical studies using combination therapies, including gene therapy, as well as studies in larger animal models of DMD, Hammers et al., 2016, to enable human trials. The ultimate goal is to enable human clinical trials to test TZDs' ability to alleviate disease progression as a monotherapy, which, if successful, also will allow the eventual use of TZDs in combination therapies.

All experiments to be performed for all animal studies as well as for histology assays will be blinded. In all cases, the person performing the experiments will know the experimental protocol to be performed, but not the identity of the mouse or sample other than by a code number assigned by another person who will be managing the study.

After all data is obtained, the code will be broken to allow the data to be analyzed in appropriate groups. Statistical methods. Proposed animal numbers were calculated from a power analysis (power=0.8; α=0.05) using MF profiles (JHU) or muscle function (UF) as the primary parameter. Based on previous data sets, n=6 and n=12, respectively, should be sufficient to resolve significant differences among groups with consideration of the variance imposed by the mdx pathology. Parametric data (anticipated) will be analyzed using ANOVA or two-tailed Welch's T-tests, where appropriate. Non-parametric data will be analyzed using Kruskal-Wallis or Mann-Whitney U tests. Consideration of relevant biological variables. Since DMD and BMD are X-linked diseases, only male mice will be used for this project. Mice of various ages within the life expectancy of these models will be investigated.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Hoffman, E. P., Brown, R. H., Jr. & Kunkel, L. M. Dystrophin: the protein product of the Duchenne muscular dystrophy locus. Cell 51, 919-928 (1987).
Petrof, B. J., Shrager, J. B., Stedman, H. H., Kelly, A. M. & Sweeney, H. L. Dystrophin protects the sarcolemma from stresses developed during muscle contraction. Proc Natl Acad Sci USA 90, 3710-3714 (1993).
England, S. B. et al. Very mild muscular dystrophy associated with the deletion of 46% of dystrophin. Nature 343, 180-182 (1990).
Barp, A. et al. Muscle MRI and functional outcome measures in Becker muscular dystrophy. Sci Rep 7, 16060 (2017).
Cripe, L. H. & Tobias, J. D. Cardiac considerations in the operative management of the patient with Duchenne or Becker muscular dystrophy. Paediatr Anaesth 23, 777-784 (2013).
Duan, D. Systemic AAV Micro-dystrophin Gene Therapy for Duchenne Muscular Dystrophy. Mol Ther 26, 2337-2356 (2018).
Kharraz, Y., Guerra, J., Pessina, P., Serrano, A. L. & Munoz-Canoves, P. Understanding the process of fibrosis in Duchenne muscular dystrophy. BioMed research international 2014, 965631 (2014).
De Paepe, B., Creus, K. K. & De Bleecker, J. L. The tumor necrosis factor superfamily of cytokines in the inflammatory myopathies: potential targets for therapy. Clin Dev Immunol 2012, 369432 (2012).
De Paepe, B., Creus, K. K., Martin, J. J. & De Bleecker, J. L. Upregulation of chemokines and their receptors in Duchenne muscular dystrophy: potential for attenuation of myofiber necrosis. Muscle & nerve 46, 917-925 (2012).
Patsalos, A., Tzerpos, P., Wei, X. & Nagy, L. Myeloid cell diversification during regenerative inflammation: Lessons from skeletal muscle. Semin Cell Dev Biol (2021).
Patsalos, A. et al. A growth factor-expressing macrophage subpopulation orchestrates regenerative inflammation via GDF-15. J Exp Med 219 (2022).

Martinez, F. O., Helming, L. & Gordon, S. Alternative Activation of Macrophages: An Immunologic Functional Perspective. Annual Review of Immunology 27, 451-483 (2009).
Huang, J. T. et al. Interleukin-4-dependent production of PPAR-gamma ligands in macrophages by 12/15-lipoxygenase. Nature 400, 378-382 (1999).
Varga, T. et al. Macrophage PPARgamma, a Lipid Activated Transcription Factor Controls the Growth Factor GDF3 and Skeletal Muscle Regeneration. Immunity 45, 1038-1051 (2016).
Daniel, B. et al. The Nuclear Receptor PPARgamma Controls Progressive Macrophage Polarization as a Ligand-Insensitive Epigenomic Ratchet of Transcriptional Memory. Immunity 49, 615-626 e616 (2018).
Levine, A. J. & Brivanlou, A. H. GDF3, a BMP inhibitor, regulates cell fate in stem cells and early embryos. Development 133, 209-216 (2006).
Levine, A. J., Levine, Z. J. & Brivanlou, A. H. GDF3 is a BMP inhibitor that can activate Nodal signaling only at very high doses. Developmental biology 325, 43-48 (2009).
Baron, D. et al. Immune response and mitochondrial metabolism are commonly deregulated in DMD and aging skeletal muscle. PLOS One 6, e26952 (2011).
Patsalos, A. et al. In vivo GDF3 administration abrogates aging related muscle regeneration delay following acute sterile injury. Aging Cell 17, e12815 (2018).
Lambert, J. et al. PPARgamma agonists promote the resolution of myelofibrosis in preclinical models. J Clin Invest 131 (2021).
Arnold, S. V. et al. Understanding Contemporary Use of Thiazolidinediones. Circ Heart Fail 12, e005855 (2019).
Sohda, T. et al. Studies on antidiabetic agents. II. Synthesis of 5-[4-(1-methylcyclohexylmethoxy)-benzyl]thiazolidine-2,4-dione (ADD-3878) and its derivatives. Chem Pharm Bull (Tokyo) 30, 3580-3600 (1982).
Sohda, T. et al. Studies on antidiabetic agents. I. Synthesis of 5-[4-(2-methyl-2-phenylpropoxy)-benzyl]thiazolidine-2,4-dione (AL-321) and related compounds. Chem Pharm Bull (Tokyo) 30, 3563-3573 (1982).
Sohda, T. et al. Studies on antidiabetic agents. III. 5-Arylthiazolidine-2,4-diones as potent aldose reductase inhibitors. Chem Pharm Bull (Tokyo) 30, 3601-3616 (1982).
Fujita, T. et al. Reduction of insulin resistance in obese and/or diabetic animals by 5-[4-(1-methylcyclohexylmethoxy)benzyl]-thiazolidine-2,4-dione (ADD-3878, U-63,287, ciglitazone), a new antidiabetic agent. Diabetes 32, 804-810 (1983).
Kletzien, R. F., Clarke, S. D. & Ulrich, R. G. Enhancement of adipocyte differentiation by an insulin-sensitizing agent. Mol Pharmacol 41, 393-398 (1992).
Kletzien, R. F., Foellmi, L. A., Harris, P. K., Wyse, B. M. & Clarke, S. D. Adipocyte fatty acid-binding protein: regulation of gene expression in vivo and in vitro by an insulin-sensitizing agent. Mol Pharmacol 42, 558-562 (1992).
Harris, P. K. & Kletzien, R. F. Localization of a pioglitazone response element in the adipocyte fatty acid-binding protein gene. Mol Pharmacol 45, 439-445 (1994).
Tontonoz, P., Hu, E. & Spiegelman, B. M. Stimulation of adipogenesis in fibroblasts by PPAR gamma 2, a lipid-activated transcription factor. Cell 79, 1147-1156 (1994).
Tontonoz, P. et al. Adipocyte-specific transcription factor ARF6 is a heterodimeric complex of two nuclear hormone receptors, PPAR gamma and RXR alpha. Nucleic Acids Res 22, 5628-5634 (1994).
Lehmann, J. M. et al. An antidiabetic thiazolidinedione is a high affinity ligand for peroxisome proliferator-activated receptor gamma (PPAR gamma). J Biol Chem 270, 12953-12956 (1995).
Sakamoto, J. et al. Activation of human peroxisome proliferator-activated receptor (PPAR) subtypes by pioglitazone. Biochem Biophys Res Commun 278, 704-711 (2000).
Betteridge, D. J. Effects of pioglitazone on lipid and lipoprotein metabolism. Diabetes Obes Metab 9, 640-647 (2007).
Shah, P. & Mudaliar, S. Pioglitazone: side effect and safety profile. Expert Opin Drug Saf 9, 347-354 (2010).
Lebovitz, H. E. Thiazolidinediones: the Forgotten Diabetes Medications. Curr Diab Rep 19, 151 (2019).
Devchand, P. R., Liu, T., Altman, R. B., FitzGerald, G. A. & Schadt, E. E. The Pioglitazone Trek via Human PPAR Gamma: From Discovery to a Medicine at the FDA and Beyond. Front Pharmacol 9, 1093 (2018).
Dormandy, J. A. et al. Secondary prevention of macrovascular events in patients with type 2 diabetes in the PROactive Study (PROspective pioglitAzone Clinical Trial In macro Vascular Events): a randomised controlled trial. Lancet 366, 1279-1289 (2005).
Legchenko, E. et al. PPARgamma agonist pioglitazone reverses pulmonary hypertension and prevents right heart failure via fatty acid oxidation. Sci Transl Med 10 (2018).
Soccio, R. E., Chen, E. R. & Lazar, M. A. Thiazolidinediones and the promise of insulin sensitization in type 2 diabetes. Cell Metab 20, 573-591 (2014).
Clarke, G. D. et al. Pioglitazone Improves Left Ventricular Diastolic Function in Subjects With Diabetes. Diabetes Care 40, 1530-1536, doi: 10.2337/dc17-0078 (2017).
Yotsukura, M., Miyagawa, M., Tsuya, T., Ishihara, T. & Ishikawa, K. Pulmonary hypertension in progressive muscular dystrophy of the Duchenne type. Jpn Circ J 52, 321-326 (1988).
Todd, M. K., Watt, M. J., Le, J., Hevener, A. L. & Turcotte, L. P. Thiazolidinediones enhance skeletal muscle triacylglycerol synthesis while protecting against fatty acid-induced inflammation and insulin resistance. Am J Physiol Endocrinol Metab 292, E485-493 (2007).
Chawla, A. et al. PPAR-gamma dependent and independent effects on macrophage-gene expression in lipid metabolism and inflammation. Nat Med 7, 48-52 (2001).
Ricote, M., Li, A. C., Willson, T. M., Kelly, C. J. & Glass, C. K. The peroxisome proliferator-activated receptor-gamma is a negative regulator of macrophage activation. Nature 391, 79-82 (1998).
Hammers, D. W. et al. The D2.mdx mouse as a preclinical model of the skeletal muscle pathology associated with Duchenne muscular dystrophy. Sci Rep 10, 14070 (2020).
Li, D., Yue, Y., Lai, Y., Hakim, C. H. & Duan, D. Nitrosative stress elicited by nNOSmicro delocalization inhibits muscle force in dystrophin-null mice. J Pathol 223, 88-98 (2011).
Mendell, J. R. et al. Assessment of Systemic Delivery of rAAVrh74.MHCK7.micro-dystrophin in Children With Duchenne Muscular Dystrophy: A Nonrandomized Controlled Trial. JAMA Neurol (2020).
Geissmann, F., Jung, S. & Littman, D. R. Blood monocytes consist of two principal subsets with distinct migratory properties. Immunity 19, 71-82 (2003).
Varga, T. et al. Tissue LyC6-Macrophages Are Generated in the Absence of Circulating LyC6-Monocytes and Nur77 in a Model of Muscle Regeneration. The Journal of Immunology 191, 5695-5701 (2013).
Chazaud, B. Macrophages: Supportive cells for tissue repair and regeneration. Immunobiology 219, 172-178 (2014).
Mojumdar, K. et al. Inflammatory monocytes promote progression of Duchenne muscular dystrophy and can be therapeutically targeted via CCR2. EMBO Mol Med 6, 1476-1492 (2014).
Barton, E. R., Wang, B. J., Brisson, B. K. & Sweeney, H. L. Diaphragm displays early and progressive functional deficits in dysferlin-deficient mice. Muscle & nerve 42, 22-29 (2010).
Hardy, D. et al. Comparative Study of Injury Models for Studying Muscle Regeneration in Mice. PLOS ONE 11, e0147198 (2016).
Arnold, L. et al. Inflammatory monocytes recruited after skeletal muscle injury switch into antiinflammatory macrophages to support myogenesis. The Journal of Experimental Medicine 204, 1057-1069 (2007).
Varga, T. et al. Highly Dynamic Transcriptional Signature of Distinct Macrophage Subsets during Sterile Inflammation, Resolution, and Tissue Repair. The Journal of Immunology 196, 4771-4782 (2016).
De Micheli, A. J. et al. Single-Cell Analysis of the Muscle Stem Cell Hierarchy Identifies Heterotypic Communication Signals Involved in Skeletal Muscle Regeneration. Cell Rep 30, 3583-3595 e3585 (2020).
Giordani, L. et al. High-Dimensional Single-Cell Cartography Reveals Novel Skeletal Muscle-Resident Cell Populations. Mol Cell 74, 609-621 e606 (2019).
Baitsch, D. et al. Apolipoprotein E induces antiinflammatory phenotype in macrophages. Arterioscler Thromb Vasc Biol 31, 1160-1168 (2011).
Ho, M. M., Manughian-Peter, A., Spivia, W. R., Taylor, A. & Fraser, D. A. Macrophage molecular signaling and inflammatory responses during ingestion of atherogenic lipoproteins are modulated by complement protein C1q. Atherosclerosis 253, 38-46 (2016).
Borneman, A., Kuschel, R. & Fujisawa-Sehara, A. Analysis for transcript expression of meltrin alpha in normal, regenerating, and denervated rat muscle. J Muscle Res Cell Motil 21, 475-480 (2000).
Zhang, M. J., Ntranos, V. & Tse, D. Determining sequencing depth in a single-cell RNA-seq experiment. Nat Commun 11, 774 (2020).
Yin, H., Price, F. & Rudnicki, M. A. Satellite cells and the muscle stem cell niche. Physiol Rev 93, 23-67 (2013).
Skelly, D. A. et al. Single-Cell Transcriptional Profiling Reveals Cellular Diversity and Intercommunication in the Mouse Heart. Cell Rep 22, 600-610 (2018).
Crossland, H., Constantin-Teodosiu, D. & Greenhaff, P. L. The Regulatory Roles of PPARs in Skeletal Muscle Fuel Metabolism and Inflammation: Impact of PPAR Agonism on Muscle in Chronic Disease, Contraction and Sepsis. Int J Mol Sci 22 (2021).
van den Brink, S. C. et al. Single-cell sequencing reveals dissociation-induced gene expression in tissue subpopulations. Nat Methods 14, 935-936 (2017).
Bai, D. et al. Hepatic, Metabolic, and Toxicity Evaluation of Repeated Oral Administration of SnS2 Nanoflowers in Mice. Toxicol Sci 164, 501-511 (2018).
Funk, J. A. et al. Characterization of renal toxicity in mice administered the marine biotoxin domoic Acid. J Am Soc Nephrol 25, 1187-1197 (2014).
Artaechevarria, X. et al. Longitudinal study of a mouse model of chronic pulmonary inflammation using breath hold gated micro-CT. Eur Radiol 20, 2600-2608 (2010).
De Vleeschauwer, S. I. et al. Repeated invasive lung function measurements in intubated mice: an approach for longitudinal lung research. Lab Anim 45, 81-89 (2011).
Kallunki, T., Barisic, M., Jaattela, M. & Liu, B. How to Choose the Right Inducible Gene Expression System for Mammalian Studies? Cells 8 (2019).
Raman, S. V. et al. Eplerenone for early cardiomyopathy in Duchenne muscular dystrophy: a randomised, double-blind, placebo-controlled trial. Lancet Neurol 14, 153-161 (2015).
Politano, L. et al. Development of cardiomyopathy in female carriers of Duchenne and Becker muscular dystrophies. JAMA 275, 1335-1338 (1996).
Grady, R. M. et al. Skeletal and cardiac myopathies in mice lacking utrophin and dystrophin: a model for Duchenne muscular dystrophy. Cell 90, 729-738 (1997).
Ponnusamy, S. et al. Androgen receptor agonists increase lean mass, improve cardiopulmonary functions and extend survival in preclinical models of Duchenne muscular dystrophy. Hum Mol Genet 26, 2526-2540 (2017).
Bates, J. H. & Irvin, C. G. Measuring lung function in mice: the phenotyping uncertainty principle. J Appl Physiol (1985) 94, 1297-1306 (2003).
Vinhas, M., Araujo, A. C., Ribeiro, S., Rosario, L. B. & Belo, J. A. Transthoracic echocardiography reference values in juvenile and adult 129/Sv mice. Cardiovasc Ultrasound 11, 12 (2013).
Hammers, D. W. et al. Tadalafil Treatment Delays the Onset of Cardiomyopathy in Dystrophin-Deficient Hearts. J Am Heart Assoc 5 (2016).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating a disease, condition, or disorder associated with impaired muscle regeneration, the method comprising administering a PPARγ agonist to a subject in need of treatment thereof.

2. The method of claim 1, wherein the PPARγ agonist comprises one or more thiazolidinediones.

3. The method of claim 2, wherein the one or more thiazolidinediones is selected from pioglitazone and rosiglitazone.

4. The method of claim 1, wherein the disease, condition, or disorder associated with impaired muscle regeneration is selected from a muscular dystrophy, an inflammatory muscle disease, trauma or injury, and aging.

5. The method of claim 4, wherein the muscular dystrophy is selected from Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD) (also known as Landouzy-Dejerine), myotonic mystrophy (MMD) (also known as Steinert's Disease), oculopharyngeal muscular dystrophy (OPMD), distal muscular dystrophy (DD), and congenital muscular dystrophy (CMD).

6. The method of claim 4, wherein the inflammatory muscle disease is selected from polymyositis, dermatomyositis, inclusion body myositis, juvenile myositis, and necrotizing autoimmune myopathy.

7. The method of claim 1, wherein the disease, condition, or disorder is at an early stage of disease progression.

8. The method of claim 1, wherein the disease, condition, or disorder is at a late stage of disease progression.

9. The method of claim 1, wherein administrating the PPARγ agonist improves one or more of muscle function, muscle structure, muscle fiber cross-sectional area, muscle regeneration, and cardiopulmonary function of the subject.

10. The method of claim 1, wherein administering the PPARγ agonist ameliorates or attenuates one or more of disease progression, fibrosis, necrosis of muscle fiber, and inflammation of the subject.

11. The method of claim 1, wherein administering the PPARγ agonist enhances regenerative macrophage activity in the subject.

12. The method of claim 1, wherein administering the PPARγ agonist promotes a macrophage phenotype transition from pro-inflammatory to pro-regenerative.

13. The method of claim 1, wherein the subject has an age of less than about 5 years, between about 5 years to about 12 years old, between about 12 years to about 15 years, and greater than 15 years.
